(19) United States
(12) Patent Application Publication
(10) Pub. No.: US 2015/0366806 A1
(43) Pub. Date: Dec. 24, 2015

(54) Method of Engineering Nanoparticle

(71) Applicant: Vuong Trieu, Agoura Hills, CA (US)

(72) Inventor: Vuong Trieu, Agoura Hills, CA (US)

(21) Appl. No.: 14/246,012

(22) Filed: Apr. 4, 2014

Related U.S. Application Data
(63) Continuation of application No. 61/853,562, filed on Apr. 8, 2013.

(51) Int. Cl. A61K 9/14; A61K 47/34; A61K 31/337

(52) U.S. Cl. CPC A61K 9/146; A61K 31/337; A61K 47/34

(57) ABSTRACT

The present invention relates to methods to guide the engineering of nanoparticle drugs for intravenous administration based on various pharmacokinetic parameters and other tests. The methods of the present invention have particular use in formulating nanoparticles containing cytotoxic drugs for the treatment of cancer. The guiding principles are properties which facilitate the release of drugs into the patient including unstable in plasma/blood, low AUC, low Cₘₐₓ, high Vd, CMC above experimental Cmax of the drug, high tumor/plasma AUC. The present invention also provides for methods of administration and compositions which are unstable after administration to a patient so that the cytotoxic drug may bind to endogenous drug transporters and be delivered to tumors in the patient.

### CROSS-REFERENCE TO RELATED APPLICATIONS

This application claims the benefit of priority to U.S. Provisional Application No. 61/853,562 filed Apr. 8, 2013 which is incorporated by reference in its entirety.

### STATEMENT REGARDING FEDERALLY SPONSORED RESEARCH OR DEVELOPMENT

Not applicable.

### FIELD OF THE INVENTION

The present invention relates to methods of identifying clinically effective nanoparticle drugs for intravenous administration based on various pharmacokinetic parameters and other tests. The methods of the present invention have particular use in engineering nanoparticles containing cytotoxic drugs for the treatment of cancer. The compositions identified by using the methods of the present invention may have properties which facilitate the release of drugs into the patient including unstable in plasma/blood, low AUC, low Cₘₐₓ, high Vd, CMC above Cₘₐₓ of the drug, high tumor/plasma AUC.

### BACKGROUND OF THE INVENTION

Recent years have witnessed unprecedented growth of research and applications in the area of nanoscience and nanotechnology. There is increasing optimism that nanotechnology, as applied to medicine, will bring significant advances in the diagnosis and treatment of disease. Anticipated applications in medicine include drug delivery, both in vitro and in vivo diagnostics, nutraceuticals and production of improved biocompatible materials. Currently many substances are under investigation for drug delivery and more specifically for cancer therapy. Interestingly pharmaceutical sciences are using nanoparticles to reduce toxicity and side effects of drugs. Many drugs used to treat patients are administered by an intravenous route.

Abraxane™ and Taxol™ are chemotherapeutic drugs. Both drugs are used to treat breast cancer. These cytotoxic medicines arrest the growth of cells in case of cancerous tissues. They essentially differ in the excipients they carry and their effectiveness. Paclitaxel is an antineoplastic drug used in chemotherapy. It is an alkaloid derived from plants and prevents microtubule formation in cells. The drug is solvent based and should be carefully administered since it is an irritant. The dosage and duration of administration of drug depends on the Body Mass Index. Side effects of Taxol include bone marrow suppression (primarily neutropenia), hair loss, arthralgias and myalgias, pain in the joints and muscles, peripheral neuropathy, nausea and vomiting, diarrhea, mouth sores, and hypersensitivity reaction, which can be dose limiting.

Abraxane is paclitaxel formulated as albumin encapsulated nanoparticles. The Abraxane formulation is free of the solvent—Cremophor™—that is present in Taxol. The absence of solvent allows the paclitaxel to bind to endogenous drug transporters and be transported by proteins such as albumin mediated transport mechanism. Protein receptors are common on the surface of tumor cells which facilitate the binding of drug molecule.

IG-001, also known as Genexol-PM™ or Paxus-PM™, is a Cremophor-free polymer-bound nanoparticle paclitaxel. Instead of using albumin to encapsulate the paclitaxel, IG-001 uses diblock mPEG-PDLLA polymer. Biodegradable polymeric micelle-type drug compositions, containing a water-soluble amphiphilic block copolymer micelle having a hydrophilic poly(alkylene oxide) component and a hydrophobic biodegradable component have been used to develop formulations in which a hydrophobic drug is physically trapped in the micelle. This micelle-type composition, enveloping a hydrophobic drug, can solubilize the hydrophobic drug in a hydrophilic environment to form a solution.

Nanomedicine is an emerging field of medicine in which drug treatments can be improved by formulating new delivery systems for drugs without using solvents found in traditional formulations However, many challenges must be overcome if the application of nanotechnology is to realize the anticipated improved understanding of the patho-physiological basis of disease, bring more sophisticated diagnostic opportunities, and yield improved therapies. New compositions and criteria for formulating nanoparticles which can be used for intravenous administration with a variety of drug components are essential to the progress of nanomedicine.

### SUMMARY OF THE INVENTION

The present invention relates to methods of identifying clinically effective nanoparticle formulated drugs used for administration, particularly intravenous administration. The methods of the present invention can be utilized as guidance for engineering into the nanoparticle desired properties. The methods of the present invention provide drugs entrained by nanoparticles which have properties which are counterintuitive when compared to traditional formulations. For example, methods to develop effective nanoparticle formulations for intravenous administration of drugs for the treatment of patients require the determination of stability of the drug in plasma/blood, of the mechanism and rate of drug release, of Cₘₐₓ, AUC and Vd so that a profile of the drug might be obtained. The methods of the present invention provide for iterative e testing of nanoparticles to yield nanoparticle formulations with a high chance of clinical success.

The present invention relates to methods of identifying clinically effective nanoparticle formulations for effective treatment as an intravenous drug formulation for treatment of a human patient by determining the experimental Cₘₐₓ and Critical Micelle Concentration (CMC) of a given drug formulation. One of the desired outcomes of the nanoparticles are higher CMC than Cmax.

The present invention relates to methods of identifying clinically effective nanoparticle formulations for an effective treatment as an intravenous drug formulation for treatment of a human patient by determining the pharmacokinetic parameters of the drug and making a nanoparticle formulation which optimizes the pharmacokinetic parameters to provide an effective treatment. Examples of pharmacokinetic parameters of the drug are Cₘₐₓ, AUC, and Vd.

The present invention relates to methods of identifying clinically effective nanoparticle formulation for an effective treatment as an intravenous drug formulation for treatment of a human patient by determining the release profile of a neat drug. The desired outcome would be release that is equal to or better than neat drug.

The present invention relates to methods of identifying clinically effective cancer treatment drug compositions comprising a cancer treatment drug entrained in a nanoparticle wherein the composition is has a critical micelle concentration (CMC) higher than the Cₘₐₓ.

The present invention relates to methods of identifying clinically effective cytotoxic drug compositions comprising a cytotoxic drug entrained in a nanoparticle wherein the composition is has a critical micelles concentration (CMC) higher than the Cₘₐₓ.

The present invention relates to methods of identifying clinically effective cancer treatment drug compositions comprising cancer treatment drugs entrained in a where the composition has a CMC higher than the Cₘₐₓ and where the composition may be bound to and transported by endogenous drug transporters in a mammal.

The present invention relates to methods of identifying clinically effective a cytotoxic drug composition comprising a cytotoxic drug entrained in a where the composition has a CMC higher than the Cₘₐₓ and where the composition may be bound to and transported by endogenous drug transporters in a mammal.

The present invention relates to methods of identifying clinically effective cancer treatment drug compositions comprising cancer treatment drugs encapsulated in a diblock copolymer wherein the composition is has a critical micelles concentration (CMC) higher than the Cₘₐₓ.

The present invention relates to methods of identifying clinically effective cytotoxic drug compositions comprising a cytotoxic drug encapsulated in a diblock copolymer wherein the composition is has a critical micelle concentration (CMC) higher than the Cₘₐₓ.

The present invention relates to methods of identifying clinically effective cancer treatment compositions comprising cancer treatment drugs encapsulated in a diblock copolymer where the composition has a CMC higher than the Cₘₐₓ and where the composition may be bound to and transported by endogenous drug transporters in a mammal.

The present invention relates to methods of identifying clinically effective cytotoxic drug composition comprising a cytotoxic drug encapsulated in a diblock copolymer where the composition has a CMC higher than the experimental Cₘₐₓ and where the composition may be bound to and transported by endogenous drug transporters in a mammal.

The present invention also related to methods of identifying clinically effective cancer treatment drug compositions comprising cancer treatment drugs entrained in a nanoparticle where the composition has a low AUC and Cₘₐₓ and high Vd relative to solvent-based formulation such that the composition is bound to and transported by endogenous drug transporters when administered to a human.

The present invention also related to methods of identifying clinically effective cytotoxic drug compositions comprising cytotoxic drug entrained in a nanoparticle where the composition has a low AUC and C_{Max} and high Vd relative to solvent-based formulation such that the composition is bound to and transported by endogenous drug transporters when administered to a human.

The present invention also related to methods of developing cancer treatment drug compositions comprising cancer treatment drugs encapsulated in a diblock copolymer where the composition has a low AUC and C_{Max} and high Vd relative to solvent based formulation such that the composition is bound to and transported by endogenous drug transporters when administered to a human.

The present invention also related to methods of developing cytotoxic drug compositions comprising cytotoxic drug encapsulated in a diblock copolymer where the composition has a low AUC and C_{Max} and high Vd relative to solvent based formulation such that the composition is bound to and transported by endogenous drug transporters when administered to a human.

The present invention relates to methods of developing a nanoparticle formulations for an effective treatment as an intravenous drug formulation for treatment of a human patient comprising determining the release profile of a the drug in a given formulation and making a nanoparticle formulation which releases the drug at a rate that is faster than the given drug.

### BRIEF DESCRIPTION OF THE FIGURES

FIG. 1. Nanoparticles of various stabilities

FIG. 2. Plot of Net (Δ) Overall Response Rate (ΔORR) of phase 3 data for IG-001 (interim), Abraxane™, and Tocosol™.

FIG. 3a. Plot of particle size versus paclitaxel concentration for nab-paclitaxel in phosphate buffered saline (PBS) and 0.1× serum and 1× serum

FIG. 3b. Plot of particle size versus paclitaxel concentration for IG-001 in phosphate buffered saline (PBS) and 0.1× serum and 1× serum

FIG. 4a. Paclitaxel release from various formulations

FIG. 4b. Paclitaxel release evaluated by Rapid Equilibrium Dialysis

FIG. 5a. Dose-proportionality graph for IG-001, IG-002, Taxol™ and Abraxane

FIG. 5b. Volume of distribution graph for IG-001, IG-002, Taxol, and Abraxane

FIG. 6. Tumor distribution for IG-002, Taxol and Abraxane in MDA-MB-435 xenograft model.

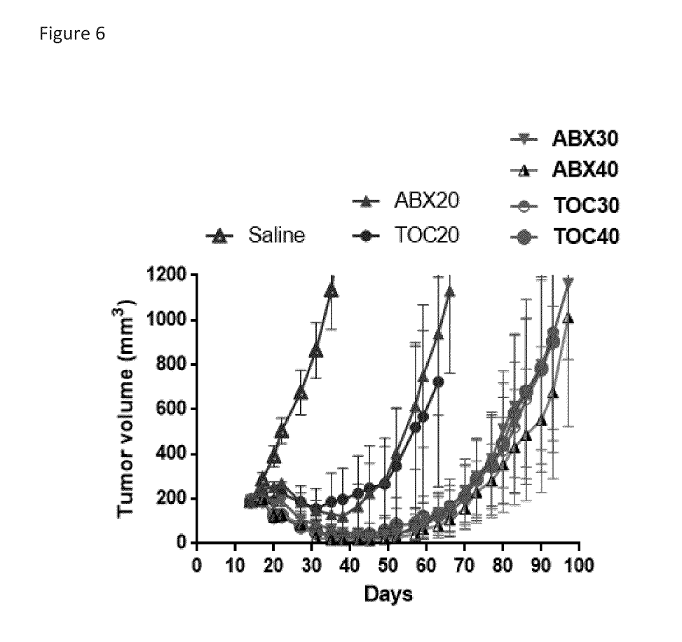

FIG. 7. Plot of tumor growth inhibition ratio (T/C) for IG-001 versus T/C for Taxol for various tumor types

FIG. 8. Plot of tumor volume versus time for SKOV3 (ovarian cancer) when treated with IG-001, Taxol and control.

FIG. 9. Plot of tumor volume versus time for DLD-1 (colon cancer) when treated with IG-001, Taxol and control.

FIG. 10. Plot of tumor volume versus time for NIH H1299 (lung cancer) when treated with IG-001, Taxol and control.

FIG. 11. Plot of tumor volume versus time for AsPC-1 (pancreatic cancer) when treated with IG-001, Abraxane, gemcitabine and control.

FIG. 12. Plot of tumor volume versus time for PANC-1 (pancreatic cancer) when treated with IG-001 (20 mg/kg), IG-001 (50 mg/kg), Taxol (20 mg/kg), gemcitabine (140 mg/kg) and control.

FIG. 13. Plot of tumor volume versus time for PaCa-2 (early pancreatic cancer) when treated with IG-001 (25 mg/kg), IG-001 (40 mg/kg), IG-001 (60 mg/kg), Taxol (25 mg/kg), gemcitabine (140 mg/kg) and control

### DETAILED DESCRIPTION OF THE INVENTION

The present invention relates to methods of determining nanoparticle engineered to achieve clinically effective nanoparticle formulated drugs used for intravenous administration. The properties of the formulated nanoparticles, including pharmacokinetic (pK) parameters can be engineered to achieve desired treatment outcomes. The methods of the present invention provide drugs entrained by nanoparticles which have properties which may be counterintuitive when compared to traditional teachings. Methods to develop effective nanoparticle formulations for intravenous administration of drugs for the treatment of patients may require the determination of the stability of the drug in plasma/blood and/or of the mechanism of drug release and/or the rate of drug release, and pharmacokinetic profile that permits effective drug treatment for the patient by determination of appropriate Cₘₐₓ, AUC and Vd.

Pharmacokinetics describes, quantitatively, the various steps of drug distribution in the body including the absorption of drugs, distribution of drugs to various organs and the elimination of drugs from the body. Various pharmacokinetic (PK) parameters include maximum observed plasma concentration (Cₘₐₓ), areas under the plasma concentration-time curve (AUCₗₐₛₜ and AUC_{inf}), areas under the first moment curve (AUMCₗₐₛₜ and AUMC_{inf}), time-to-maximum observed plasma concentration (Tₘₐₓ), half-life (T_{1/2}), the apparent terminal elimination rate constant (λ_{z}), and mean resident time (MRT).

Cₘₐₓ refers to the maximum concentration that a drug achieves in tested area after the drug has been administered. The Area Under the Curve (AUC) is a plot of concentration of drug in blood plasma against time. The area is computed from the time the drug is administered to the point where concentration in plasma is negligible. The Volume of Distribution (Vd) relates the amount of drug in the body to the measured concentration in the plasma. A large volume of distribution indicates that the drug distributes extensively into body tissues and fluids. A discussion of various pharmacokinetic parameters and the methods of measuring them can be found in Clinical Pharmacokinetics and Pharmacodynamics: Concepts and Applications, M. Rowland and T. N. Tozer, (Lippincott, Williams & Wilkins, 2010).

Polymeric micelles and nanoparticles have been used in the delivery of various drugs. Micelle stability is influenced by various factors depending on the media environment including polymer concentration, molecular mass of the core-forming block, drug incorporation, other proteins and/or cells found in serum or blood. Stability of micelles depends on the polymer concentration. Polymer micelles have a critical micelle concentration (CMC) that is the lowest concentration of polymers to produce a micelle structure. Thus, micelles form when the concentration of the surfactant is greater than the critical micelle concentration and the temperature of the system is greater than the critical micelle temperature. Micelles can form spontaneously because of the balance between entropy an enthalpy. In aqueous systems, the hydrophobic effect is the driving force for micelle formulation and surfactant molecules assembling reduce the entropy. As the concentration of the lipid increases, the unfavorable entropy considerations from the hydrophobic end of the molecule prevail. At this point the lipid hydrocarbon chains of a portion of the lipids must be sequestered away from the water. Therefore, the lipid starts to form micelles. When surfactants are present above the CMC, they can act as emulsifiers that will allow a compound that is normally insoluble to dissolve. The CMC may be determined by a variety of methods including but not limited to: 1) spectroscopic measurements using a fluorescence probe, an absorbance dye 2 and other probes; 2) electrochemical measurement using electrophoresis or capillary electrophoresis; 3) surface tension measurements and contact angle measurement; 4) optical measurements using light scattering, optical fibers and refraction; 5) other methods such as ITC, chromatography, ultrasonic velocity and others. One method for determining CMC is by particle dissolution. Starting with a certain concentration (e.g. 5 mg/ml), the drug is serially diluted in a testing matrix (PBS, blood, plasma, etc.) and the size of the nanoparticle is determined by DLS. The concentration at which the nanoparticles disappear is the CMC.

It is commonly believed that effective intravenous nanoparticle formulations should be stable in blood. In other words, effective intravenous nanoparticle formulations would result in high blood/plasma levels of stable nanoparticle (nanoparticle with CMC below its Cₘₐₓ in blood/plasma and slow release of drug), and would thereby be effective in cancer treatments. However, the compositions and methods of the present invention unexpectedly indicate that effective nanoparticle formulations for intravenous administration of drugs, especially cytotoxic drugs, are unstable in plasma/blood, provide for rapid drug release, and exhibit low Cₘₐₓ, AUC and high Vd: characteristics of rapid tissue penetration. The nanoparticle formulations of the present invention may be more effective or equally effective as conventional solvent based formulations of the same drug at equal dosing.

The prior art teaches methods to prepare sustained release micelles in which polymers with very low CMC (<0.1 μg/ml) which can be used for prolonging the circulation time before the micelle degrades. Upon intravenous injection, the micelles undergo dilution in the body. If the CMC of the micelles is high, the concentration of the polymer or surfactant falls below the CMC upon dilution and hence, the micelles dissociate. Therefore, the prior art teaches that a higher concentration of the polymer or surfactant has to be used to prepare the micelles so that they withstand the dilution of up to 5 L in the blood. However, the use of high concentrations of polymer or surfactant might not be feasible due to toxicity related dose limitations. If the polymer or surfactant has a CMC lower than 0.1 μg/ml concentrations, as low as 5 mg/ml, may be used to prepare a micelle formulations in order to counter the dilution effects in the blood. A variety of polymers including diblock copolymers, triblock copolymers and graft copolymers have been synthesized for this purpose. Thus, the prior art teaches that the nanoparticles should be crafted to be stable even after intravenous administration. The methods of the present invention provide methodologies for constructing micelle formulations in which the nanoparticles are less stable once administered such that the drug compound can be released from the nanoparticle and made available to the endogenous drug transporters delivery system. In one embodiment of the present invention it is desirable that the nanoparticles have CMC values which are higher than the Cₘₐₓ of the composition once delivered to a patient. Stable nanoparticles pharmacokinetics would be indicated by a higher Cₘₐₓ and higher AUC than solvent based paclitaxel, while the unstable nanoparticle pharmacokinetics would be indicated by a lower Cₘₐₓ and lower AUC than solvent based paclitaxel.

The present invention relates to methods of identifying clinically effective nanoparticle formulations for effective intravenous drug formulations for treatment of a human patient by determining the release profile of a neat drug and/or determining the experimental Cₘₐₓ of the drug and/or determining the pharmacokinetic parameters of the drug, and making a nanoparticle formulation which can release the drug at a rate similar to the neat drug and/or has a Critical Micelle Concentration (CMC) above the experimental Cₘₐₓ and/or optimizes the pharmacokinetic parameters to provide a clinically effective formulation. A “neat drug” is defined here as a drug that is dissolved in a solvent for administration. In a research setting, the drug can be reconstituted in acetonitrile and used directly. Acetonitrile would not be possible for human administration but it permits reconstitution of the drug for testing. In determining the experimental Cₘₐₓ nonclincally, the drug may be administered to a test animal such as dogs or monkeys so that infusion can be controlled. Blood is drawn at various time intervals, and the level of administered drug (e.g. paclitaxel) in the blood is determined. The AUC is calculated using concentration across all time points, and Cₘₐₓ is the maximum concentration. Usually the Cₘₐₓ occurs at a time point that is at end of infusion stop. In a clinical setting, infusions normally range from 30 minutes to 3 hours. Normally, the faster the infusion, the higher the Cₘₐₓ.

In the nanoparticles of the present invention the CMC of the nanoparticles may be at least 10% higher than the expected Cₘₐₓ of the nanoparticle composition. In the nanoparticles of the present invention the CMC of the nanoparticles may be at least 20% higher or 25% higher or 30% higher or 35% higher or 40% higher or 45% higher or 50% higher or 55% higher or 60% higher or 65% higher or 70% higher or 75% higher or 80% higher or 85% higher or 90% higher or 95% higher or 100% higher or 125% higher or 150% higher or 175% higher or 200% higher or 500% than the expected Cₘₐₓ of the nanoparticle composition. In the nanoparticles of the present invention the CMC of the nanoparticles may be between about 10% higher to about 250% higher or about 10% higher to about 150% higher or about 10% higher to about 125% higher or about 10% higher to about 100% higher or from about 10% higher to about 90% higher or from about 10% higher to about 80% higher or from about 10% higher to about 70% higher or from about 10% higher to about 60% higher or from about 10% higher to about 50% higher or from about 10% higher to about 40% higher or from about 10% higher to about 30% higher or about 10% higher to about 20% higher or about 20% higher to about 125% higher or about 20% higher to about 100% higher or from about 20% higher to about 90% higher or from about 20% higher to about 80% higher or from about 20% higher to about 70% higher or from about 20% higher to about 60% higher or from about 20% higher to about 50% higher or from about 20% higher to about 40% higher or from about 50% higher to about 250% or from about 50% higher to about 125% higher or from about 50% higher to about 100% higher than the expected Cₘₐₓ of the nanoparticle composition.

The present invention provides methodologies for constructing nanoparticles which ideally release their contents in vivo but are stable in an IV bag, in an infusion solution or in a reconstitute vial.

The invention provides methodologies for constructing nanoparticles which have low AUC and low Cₘₐₓ. In some embodiments the AUC of the nanoparticles of the present invention are at least 5% or at least 10% or at least 20% or at least 30% or at least 40% or at least 50% or at least 60% or at least 70% or at least 80% or at least 90% or at least 100% less than comparable solvent based formulations of the same drug. In some embodiments the AUC of the nanoparticles of the present invention are between about 5% to about 100% or from about 5% to about 75% or from about 5% to about 50% or from about 5% to about 25% or from about 5% to about 10% or from about 10% to about 75% or from about 10% to about 50% or from about 10% to about 25% or from about 25% to about 100% or from about 25% to about 75% or from about 25% to about 50% less than comparable solvent based formulations.

The present invention provides methodologies for constructing nanoparticles in which the Vd of the nanoparticles may be at least 20% higher or 25% higher or 30% higher or 35% higher or 40% higher or 45% higher or 50% higher or 55% higher or 60% higher or 65% higher or 70% higher or 75% higher or 80% higher or 85% higher or 90% higher or 95% higher or 100% higher or 125% higher or 150% higher or 175% higher or 200% higher or 500% than the Vd of comparable solvent based formulations. In the nanoparticles of the present invention the Vd of the nanoparticles may be between about 10% higher to about 250% higher or about 10% higher to about 150% higher or about 10% higher to about 125% higher or about 10% higher to about 100% higher or from about 10% higher to about 90% higher or from about 10% higher to about 80% higher or from about 10% higher to about 70% higher or from about 10% higher to about 60% higher or from about 10% higher to about 50% higher or from about 10% higher to about 40% higher or from about 10% higher to about 30% higher or about 10% higher to about 20% higher or about 20% higher to about 125% higher or about 20% higher to about 100% higher or from about 20% higher to about 90% higher or from about 20% higher to about 80% higher or from about 20% higher to about 70% higher or from about 20% higher to about 60% higher or from about 20% higher to about 50% higher or from about 20% higher to about 40% higher or from about 50% higher to about 250% or from about 50% higher to about 125% higher or from about 50% higher to about 100% higher than comparable solvent based formulations of the same drug.

The present invention relates to methods of developing a nanoparticle formulations for an effective treatment as an intravenous drug formulation for treatment of a human patient comprising determining the release profile of a the drug in a given formulation and making a nanoparticle formulation which releases the drug at a rate that is faster than the given drug. In some circumstances the effectiveness of a drug may be determined by the rate the drug is released into the body, for example, into the blood. In that case, it is desirable to formulate a composition such that the composition releases the drug into the system faster than the currently available drug formulations. For example, it is desirable to release paclitaxel into the system rapidly as this facilitates the binding of serum albumin to paclitaxel and uptake of paclitaxel by tumors. High circulating levels of paclitaxel which are not taken into the tumor can increase toxicity of the drug. The methods of the present invention provide a mechanism for increasing the rate at which the drug is released so as to facilitate the drug's uptake. In certain embodiments of the methods of the present invention the nanoparticles are designed so as to release the drug rapidly such that the drug is released fifty times, or twenty five times, or ten times, or nine times, or eight times, or seven times, or six times, or five times, or four times or three times, or two times, or 1.9 times or 1.8 times, or 1.7 times, or 1.6 times, or 1.5 times or 1.4 times or 1.3 times or 1.2 times or 1.1 times faster than other formulations of the drug, in particular formulations of the drug in various solvents. The methods of the present invention provide a mechanism for increasing the rate at which the drug is released so as to facilitate the drug's uptake. In certain embodiments of the methods of the present invention the nanoparticles are designed so as to release the drug rapidly such that the drug is released one to fifty times, or one to twenty five times, or one to ten times, or one to nine times, or one to eight times, or one to seven times, or one to six times, or one to five times, or one to four times or one to three times, or one to two times, or one to 1.9 times or one to 1.8 times, or one to 1.7 times, or one to 1.6 times, or one to 1.5 times, or one to 1.4 times or one to 1.3 times or one to 1.2 times or one to 1.1 times, 1.5 to fifty times, or 1.5 to twenty five times, or 1.5 to ten times, or 1.5 to nine times, or 1.5 to eight times, or 1.5 to seven times, or 1.5 to six times, or 1.5 to five times, or 1.5 to four times or 1.5 to three times, or 1.5 to two times, or two to fifty times, or two to twenty five times, or two to ten times, or two to nine times, or two to eight times, or two to seven times, or two to six times, or two to five times, or two to four times or two to three times, or five to fifty times, or five to twenty five times, or five to ten times, or five to nine times, or five to eight times, or five to seven times, or five to six times, faster than other formulations of the drug, in particular formulations of the drug in various solvents.

In certain embodiments of the methods of the present invention the nanoparticles are designed so as to release the paclitaxel rapidly such that the paclitaxel is released fifty times, or twenty five times, or ten times, or nine times, or eight times, or seven times, or six times, or five times, or four times or three times, or two times, or 1.9 times or 1.8 times, or 1.7 times, or 1.6 times, or 1.5 times or 1.4 times or 1.3 times or 1.2 times or 1.1 times faster than other formulations of the drug, in particular formulations of the drug in various solvents such as paclitaxel in Cremphor EL. The methods of the present invention provide a mechanism for increasing the rate at which the paclitaxel is released so as to facilitate its uptake. In certain embodiments of the methods of the present invention the nanoparticles are designed so as to release the paclitaxel rapidly such that the it is released one to fifty times, or one to twenty five times, or one to ten times, or one to nine times, or one to eight times, or one to seven times, or one to six times, or one to five times, or one to four times or one to three times, or one to two times, or one to 1.9 times or one to 1.8 times, or one to 1.7 times, or one to 1.6 times, or one to 1.5 times, or one to 1.4 times or one to 1.3 times or one to 1.2 times or one to 1.1 times, 1.5 to fifty times, or 1.5 to twenty five times, or 1.5 to ten times, or 1.5 to nine times, or 1.5 to eight times, or 1.5 to seven times, or 1.5 to six times, or 1.5 to five times, or 1.5 to four times or 1.5 to three times, or 1.5 to two times, or two to fifty times, or two to twenty five times, or two to ten times, or two to nine times, or two to eight times, or two to seven times, or two to six times, or two to five times, or two to four times or two to three times, or five to fifty times, or five to twenty five times, or five to ten times, or five to nine times, or five to eight times, or five to seven times, or five to six times, faster than other formulations of paclitaxel, in particular formulations of paclitaxel in various solvents such as Cremophor EL.

The methods of the present invention also relate to increasing the Overall Response Rate (ORR) of a given drug by identifying nanoparticle compositions that contain the drug that are more clinically effective than the drug alone or other drug formulations. ORR is defined as the proportion of patients whose best overall response is either complete response (CR) or partial response (PR) according to the standard called “Response Evaluation Criteria in Solid Tumors” (RECIST), and can be a measure of “effectiveness” of a drug.

The pharmacokinetic parameters including the rate of drug release can be altered by a variety of factors, including but not limited to the type and amount of materials used to construct the nanoparticle compositions, added excipients, drug loading, and various solvents. This invention related the stability of the nanoparticle to its stability in vivo and therefore inability to exit the blood compartment into the underlying tissues including the target tissues. The tests to define the clinically effective formulation centered around define factor which would predict in vivo instability. These to include, dissolution assays, release assays, pharmacokinetic assays.

Various formulations of nanoparticles are contemplated by the methods of the present invention. Nanoparticles include but are not limited to dendrimers, polymer micelles, niosomes, nanogels, solid lipid nanoparticles, lipid nanostructured systems, cubosomes, liposomes, peptide nanotubules, metal colloids, carbon nanotubules, fullerenes, gold nanoparticles, gold nanoshells, silicon nanoparticles and magnetic colloids.

The nanoparticles of the present invention include colloidal dispersion systems which include macromolecule complexes, nanocapsules, microspheres, beads, and lipid-based systems including oil-in-water emulsions, micelles, mixed micelles, and liposomes. Liposomes, which are artificial membrane vesicles, are useful as delivery vehicles in vivo. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidyl choline. Block polymers may be useful in formulating the nanoparticles of the present invention. One example is block copolymers with cyclodextrins which provide drug delivery as supramolecular polymeric micelles. This involves non-covalent interactions between a macromolecular polymer, which works as a host, and a small polymer molecule, which works as a guest. Triblock copolymer micelles are flower-like micelles can be formed with a triblock copolymer with small hydrophobic ends and a long hydrophilic midsection. When dissolved in water, such polymer molecules assemble to form flower-like micellar structure. These flower-like micelles can dissolve the drug in the hydrophobic core. Drug release is faster with crystalline PLA blocks than amorphous PLA blocks, possibly because crystalline PLA stacks together, leaving the drug largely at the periphery while amorphous PLA might better integrate/disperse the drug within the polymer matrix. Most micelle-forming polymers are first dissolved in organic solvent followed by addition to an aqueous medium to form micelles. The use of organic solvents can be avoided for some triblock copolymer micelles. Furthermore, through suitable selection of polymers, greater drug loading as well as sustained drug release can be achieved. Freeze-dried micelles may be easily redispersible. Unimolecular micelles may also provide a mechanism for release of drugs. The unimolecular micelle is made out of a polymer that has several hydrophilic and hydrophobic portions in itself and forms a single molecular micelle. Lipids and PEG-like hydrophilic polymers can be conjugated to form such unimolecular micelles. One such polymer is core (laur) PEG. Multiarm block copolymers can also be used to formulate micelles. For instance star-shaped or multiarmed micelles can be formed with an amphiphilic block copolymer with multiple hydrophilic blocks and a single hydrophobic block. These polymers can form micelles if the number of arms is high enough. One such polymer is H40-PLA-mPEG. Graft polymers have recently attracted significant attention in preparing micelles. Cellulose graft polymers can be used to form micelles. The cellulose portion of the polymer can be the hydrophilic part, with any hydrophobic segment conjugated to it to form an amphiphilic graft polymer.

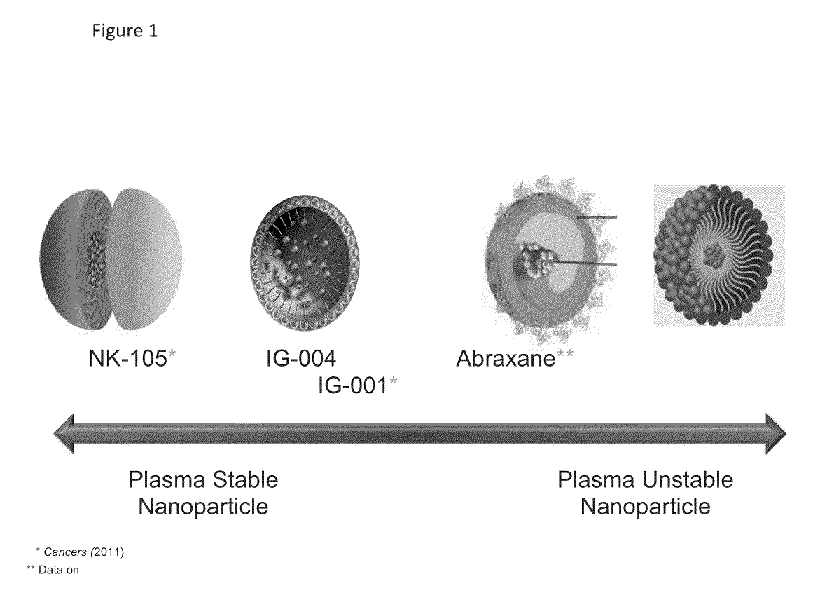

Polymers have some degree of toxicity even if they are biocompatible. Therefore, there is a need to synthesize materials that are more biocompatible for the preparation of micelles and incorporation of drugs. Oligopeptides can be very useful amphiphilic molecules for the preparation of micelles. Hydrophobic residues, such as alanine, can be used to synthesize the hydrophobic block and hydrophilic residues like histidine or lysine can be used to synthesize the hydrophilic block. Such molecules can be used as amphiphilic molecules to formulate micelles. A combination of polymer and polyamino acid can form an amphiphilic polymer. PEG—polyglutamic acid copolymer was used to prepare micelles. Micelles and nanoparticles can be of varying stabilites as shown in FIG. 1.

The polymeric micelle nanoparticle formulations include amphiphilic block copolymer which may comprise a hydrophilic block (A) and a hydrophobic block (B) linked with each other in the form of A-B, A-B-A or B-A-B structure. Additionally, the amphiphilic block copolymer may form core-shell type polymeric micelles in its aqueous solution state, wherein the hydrophobic block forms the core and the hydrophilic block forms the shell.

In one embodiment, the hydrophilic block (A) of the amphiphilic block copolymer may be polyethylene glycol (PEG) or monomethoxypolyethylene glycol (mPEG). Particularly, it may be mPEG. The hydrophilic block (A) may have a weight average molecular weight of 500-20,000 daltons, specifically 1,000-5,000 daltons, and more specifically 1,000-2,500 daltons.

The hydrophobic block (B) of the amphiphilic block copolymer may be a water-insoluble, biodegradable polymer. In one embodiment, the hydrophobic block (B) may be polylactic acid (PLA) or poly(lactic-co-glycolic acid) (PLGA). In another embodiment, the hydrophobic block (B) may have a weight average molecular weight of 500-20,000 daltons, specifically 1,000-5,000 daltons, and more specifically 1,000-2,500 daltons. Hydroxyl end groups of the hydrophobic block (B) may be protected with fatty acid groups, and particular examples of the fatty acid groups include acetate, propionate, butyrate, stearate, palmitate groups, and the like. The amphiphilic block copolymer comprising the hydrophilic block (A) and the hydrophobic block (B) may be present in the composition in an amount of 20-98 wt %, specifically 65-98 wt %, and more specifically 80-98 wt % based on the total dry weight of the composition.

In another embodiment, the hydrophilic block (A) and the hydrophobic block (B) may be present in the amphiphilic block copolymer in such a ratio that the copolymer comprises 40-70 wt %, specifically 50-60 wt % of the hydrophilic block (A) based on the weight of the copolymer. When the hydrophilic block (A) is present in a proportion less than 40%, the polymer has undesirably low solubility to water, resulting in difficulty in forming micelles. On the other hand, when the hydrophilic block (A) is present in a proportion greater than 70%, the polymer becomes too hydrophilic to form stable polymeric micelles, and thus the composition may not be used as a composition for solubilizing taxane.

A preferred paclitaxel formulation is IG-001 (also referred to as Genexol-PM™, Cynviloq™) which is a Cremophor™-free, polymeric micelle formulation of paclitaxel. IG-001 comprises biodegradable di-block copolymer composed of methoxypoly(ethyleneglycol)-poly(lactide) to form nanoparticles with a paclitaxel-containing hydrophobic core, and a hydrophilic shell. The micellar composition may be made by dissolving an amphipathic co-polymer, monomethoxypolyethylene glycol-polylactide with an average molecular weight of 1766-2000 daltons at 80° C. in ethanol. Paclitaxel is added to the dissolved co-polymer and the solution cooled to about 50° C. where room temperature water is added. Anhydrous lactose may be added and dissolved. The solution may then be filtered and lyophilized. The amount of paclitaxel in the micelle formulation can be altered. Less or more paclitaxel will change the loading % and change the CMC and properties of the formulation. The size of the nanoparticles for IG-001 is a Gaussian distribution where the mean particle size is about 10 nm to about 50 nm.

IG-002, also known as Tocosol™, is a cremophor-free, vitamin E-based paclitaxel emulsion incorporating a P-glycoprotein (Pgp) inhibitor and particle size-based tumor targeting. The particle contains three components: The inner core consists of lipophilic material; namely d,l-alpha tocopherol. At the interface between the lipophilic emulsion particle and its aqueous environment, a number of surfactants are employed, including the p-glycoprotein (pgp) inhibitor alphatocopherol polyethylene glycol succinate (TPGS). The surfactants, along with the manufacturing conditions, define and stabilize the emulsion particle size.

Cancer treatment drugs include but are not limited to cytotoxic drugs which entrained or encapsulated in the nanoparticles of the present invention and may include but are not limited to carboplatin, cisplatin, cyclophosphaminde, doxorubicin, etoposide, fluoruracil, gemcitabine, irinotecan, methotrexate, topotecan, vincristine, vinblastine, docetaxel, paclitaxel, 7-epipaclitaxel, t-acetyl paclitaxel, 10-desacetyl-paclitaxel, 10-desacetyl-7-epipaclitaxel, 7-xylosylpaclitaxel, 10-desacetyl-7-glutarylpaclitaxel, 7-N,N-dimethylglycylpaclitaxel, 7-L-alanylpaclitaxel, epothilones, rapamycin, 17-AAG or combinations thereof.

Cancer types for which the methods of the present invention may be useful include but are not limited to bladder cancer, ovarian cancer, breast cancer, pancreatic cancer, liver cancer, non-small cell lung cancer (NSCLC) and other lung cancers.

### EXAMPLES

### Example 1

### Paclitaxel Release

IG-001 (Genexol-PM) is a cremophor-free, polymeric micelle formulation of paclitaxel. IG-001 is free of Cremophor-induced toxicities such as hypersensitivity reactions, prolonged or irreversible peripheral neuropathy, and altered lipoprotein patterns. IG-001 (Genexol-PM) utilizes biodegradable di-block copolymer composed of methoxy poly(ethylene glycol)-poly(lactide) to form nanoparticles with paclitaxel containing hydrophobic core and a hydrophilic shell.

IG-002, also known as Tocosol, is a cremophor-free, vitamin E-based paclitaxel emulsion incorporating a P-glycoprotein (Pgp) inhibitor and particle size-based tumor targeting. The particle contains three components: The inner core consists of lipophilic material; d,l-alpha tocopherol. At the interface between the lipophilic emulsion particle and its aqueous environment, a number of surfactants are employed, including the p-glycoprotein (pgp) inhibitor alphatocopherol polyethylene glycol succinate (TPGS). The surfactants, along with the manufacturing conditions, define and stabilize the emulsion particle size.

Abraxane is an albumin bound nanoparticle formulation marketed by Celgene against multiple indications (Metastatic Breast Cancer and NSCLC).

NK105 is a polymeric micelle comprised of PEG-b-PAsp copolymers where the aspartate blocks are modified such that the carboxyl residues are converted to 4-phenyl-1-butanolate by an esterification reaction.

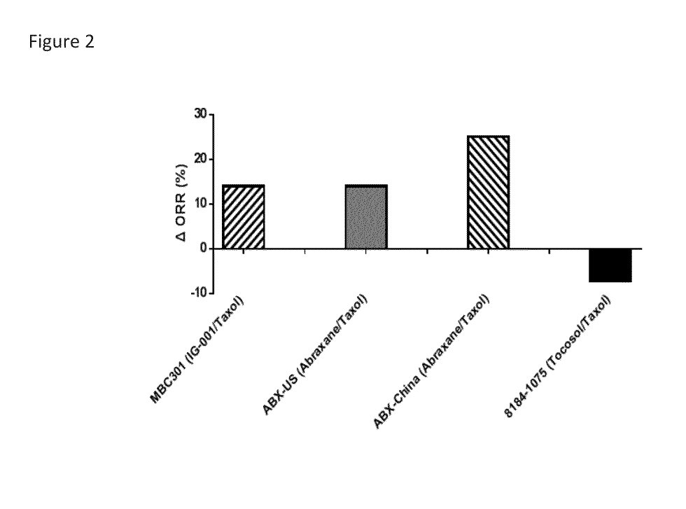

The relative stability of the four nanoparticle formulations IG-001, IG-002, ABRAXANE and NK105 are shown in FIG. 1. Clinical effectiveness of three of nanoparticle formulations is compared in FIG. 2. Briefly, in a Phase III, multicenter, randomized comparison of the safety and efficacy of weekly TOCOSOL® Paclitaxel (100 mg/m²) vs. weekly Paclitaxel Injection (80 mg/m²) in the treatment of metastatic breast cancer (MBC), the objective response rates (ORR) were assessed by RECIST in patients with MBC treated with weekly TOCOSOL™ Paclitaxel or weekly Taxol™ as first-line or second-line therapy. MBC patients (1050) were screened and a total of 821 were randomized to receive either: IG-002 (100 mg/m² weekly, IV) or Crem(aphor)-Pac(litaxel) (80 mg/m² weekly, IV) until disease progression. IG-001 phase 3 trial: Treatment: IV infusion, 3 hrs, q3w, 6 cycles; N=212 (106 per arm) with IG-001 at 300 mg/m2 and Paclitaxel (Taxol) at 175 mg/m2. Objectives: Primary: Overall Response Rate (ORR) and Secondary: OS, PFS, UP, Duration of Overall Response. Abraxane phase 3 trial (US): MBC patients were randomly assigned to receive: 3-week cycles of either ABI-007 260 mg/m2 intravenously without premedication (n=229) or standard paclitaxel 175 mg/m2 intravenously with premedication (n=225) (Gradishar et al., 2005, JCO 23: 7794-7803). Abraxane phase 3 trial (China): an open-label, multicenter study, wherein 210 patients with MBC were randomly assigned to receive: Abraxane 260 mg/m2 intravenously (i.v.) over 30 min every 3 weeks (q3w) with no premedication, or Taxol 175 mg/m2 i.v. over 3 h q3w with standard premedication (Guan et al., 2009, Asia-Pacific Journal of Clinical Oncology 5:165-174).

Both IG-001 and Abraxane demonstrated improved response rate versus Taxol; however, the stable nanoparticle formulation—IG-002/Tocosol—did not (FIG. 2).

### Example 2

### Paclitaxel Release

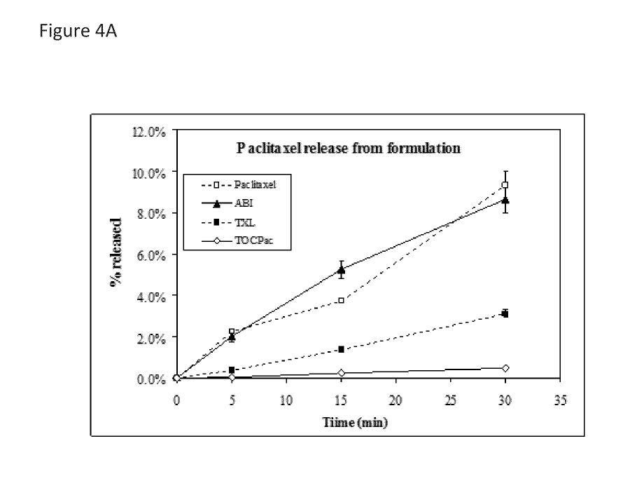

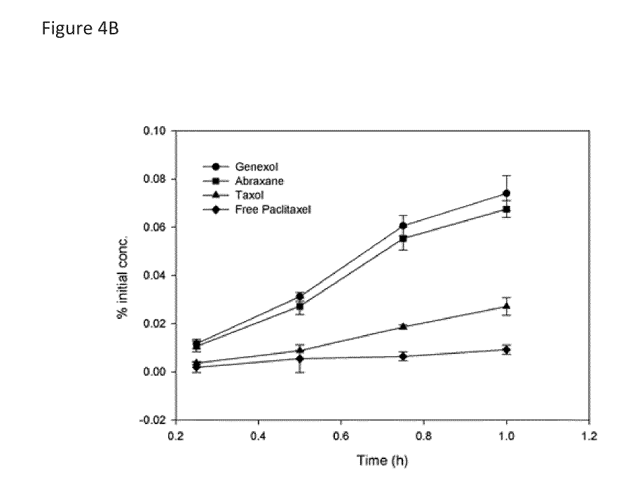

Paclitaxel release from each formulation was tested using equilibrium dialysis. Briefly, paclitaxel, IG-001, IG-002, Taxol or reconstituted Abraxane was added to one side of the well, and blank buffer to the other side. Samples were taken from the buffer side for the analysis of the appearance of free paclitaxel. The drug release profile from Abraxane appears similar to neat paclitaxel. Drug release is slowest for IG-002 (0.5% at 30 minutes, statistically significant versus the other three groups), followed by Taxol. Fast release was found for IG-001 and Abraxane. Results are shown in FIGS. 4a and b.

### Example 3

### Nanoparticle Instability

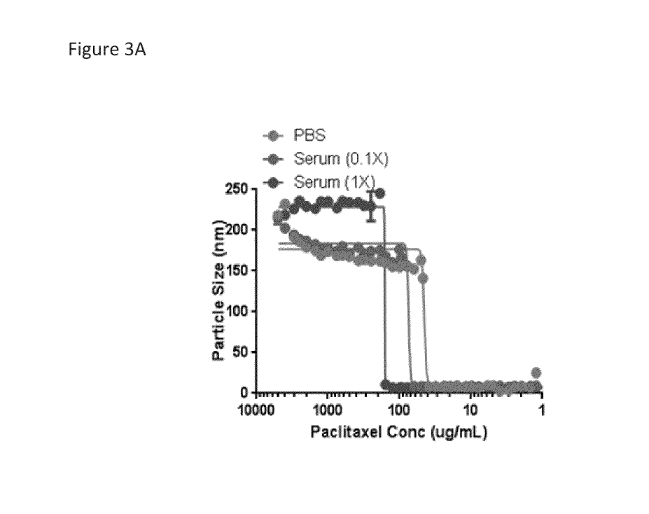

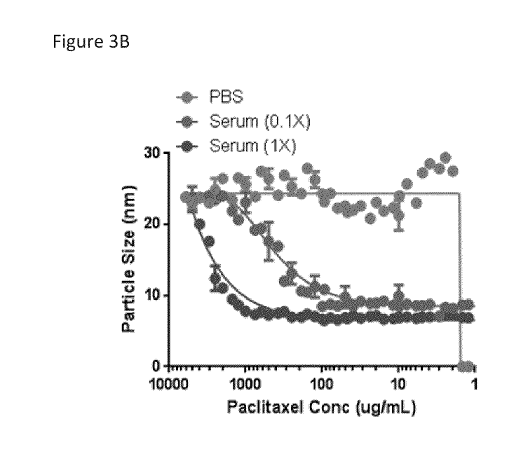

IG-001 (Genexol-PM) is a Cremophor-free, polymeric micelle formulation of paclitaxel utilizing biodegradable di-block copolymer composed of methoxy poly(ethylene glycol)-poly(lactide) to form nanoparticles with paclitaxel containing a hydrophobic core and a hydrophilic shell. IG-001 has a mean diameter of 25 nm with relatively low light scattering potential. Stability of the nanoparticle was examined across various concentrations to determine the approximate CMC—critical micelle concentration. IG-001 rapidly dissociates from intact nanoparticles upon dilution in serum at concentrations less than 50 ug/ml—higher than the Cₘₐₓ of IG-001—following a 3 hr infusion (FIGS. 3a and b). The CMC is higher than experimental maximum drug level. Therefore, once administered, IG-001 readily gives up its paclitaxel cargo to endogenous drug transporters for transport into the underlying tissues.

### Example 4

### Pharmacokinetics of Unstable Nanoparticles

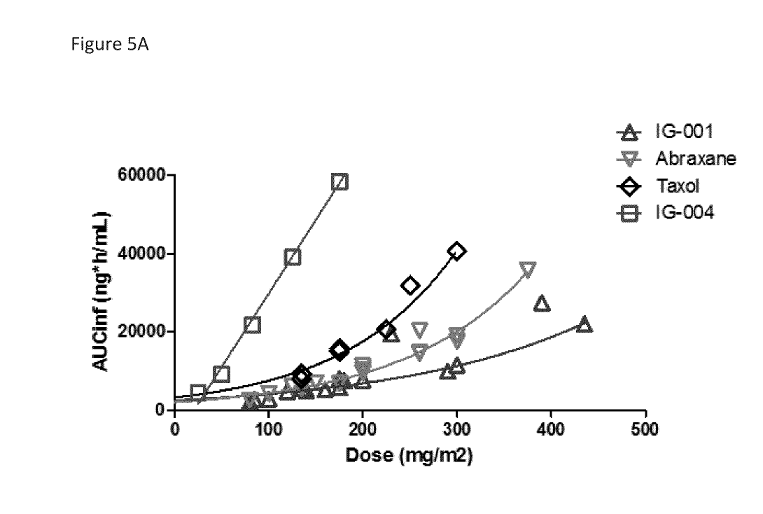

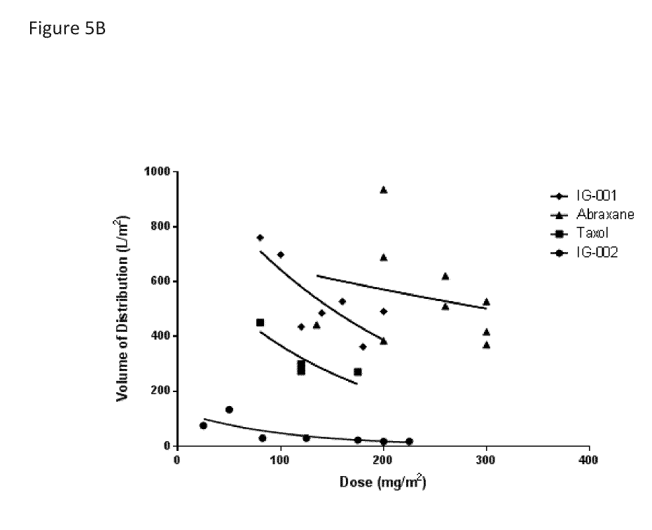

Clinical pharmacokinetics of IG-001 (Genexol-PM) was compared to Taxol and Abraxane and IG-002 (Tocosol). IG-001 range for PK dose-proportionality is the most expanded of the four paclitaxel formulations examined (Taxol, Abraxane, IG-001, IG-002) (FIG. 5a). Abraxane PK deviated from proportionality above 300 mg/m²; whereas IG-001 PK remained dose-proportional up to the highest dose of 435 mg/m². Additionally, the unstable nanoparticles IG-001 and Abraxane have lower AUC across all dose levels in comparison to the stable nanoparticle-IG-002/Tocosol.

Volume of distribution—Vd was higher for the unstable nanoparticles Abraxane and IG-001 versus stable nanoparticle (Tocosol/IG-002) or the solvent based paclitaxel formulation (Taxol). FIG. 5b.

### Example 5

### Phase II Data Including the Difficult to Perfuse Pancreatic Tumours

Clinical efficacy of IG-001 (Genexol-PM) was compared to Taxol and Abraxane across three cancer indications (MBC, NSCLC, and Pancreatic Cancer). IG-001 was more active than historical Taxol. Since Pancreatic Cancer is known to be poorly-perfused, IG-001 activity in this indication is consistent with it being able to penetrate poorly-perfused tumors.

TABLE 2
Metastatic Breast Cancer

| | | | SOLVENT BASED | | |
|---|---|---|---|---|---|
| | SOLVENT FREE | | Taxol | | |
| | IG-001 | Abraxane | 2^{nd} line 6 month | | |
| | First line | First line | recur | Taxotere | Taxotere |
| | 300 mg/m² | 260 mg/m² | 175 mg/m² | Anthrapretreated | Alkylkating agent |
| | q3w | q3w | q3w | patients | pretreated |
| ORR | 59% | 42% | 28% | 28% | 45% |
| PFS | 9M | 7M | 4.2M | 4.3M | 6.5M |
| OS | >20M | 18M | 11.7M | 11.4M | 14.7M |

ORR—overall response rate

TABLE 3
Advanced NSCLC

| | SOLVENT FREE | | | SOLVENT BASED | |
|---|---|---|---|---|---|
| | IG-001 | Abraxane | Abraxane | Taxol | Taxotere |
| | First line | First line | First line | First line | First line |
| | 230-300 mg/m² | 260 mg/m² | 100 mg/m² | 250 mg/m² | 75 mg/m² |
| | q3w + CIS | q3w mono | Weekly + carbo | q3w + CIS | q3w + CIS |
| ORR | 38% | 15.5% | 33% | 23% | 31.6% |
| PFS | 5.8M | 6M | 6.3M | 4.9M | 4.9M |
| OS | 21.7M | 11.0M | 12.1M | 10.M | 10.9M |

CIS—Cisplatin

TABLE 4
Advanced Pancreatic Cancer

| | SOLVENT FREE | | SOLVENT |
|---|---|---|---|
| | IG-001 | Abraxane | BASED |
| | 1^{st} line | 2^{nd} line | Taxol |
| | 300-435 mg/m² | 100 mg/m² | 2^{nd} line 80 mg/m² |
| | q3wks | Wkly | Wkly |
| ORR | 6.7% | 5.3% | 10% |
| PFS | 3.2M | 1.7M | |
| OS | 6.5M | 7.8M | 6.7M |

+ gemcitabine

### Example 6

### Tumor Growth Inhibition (T/C) for IG-001

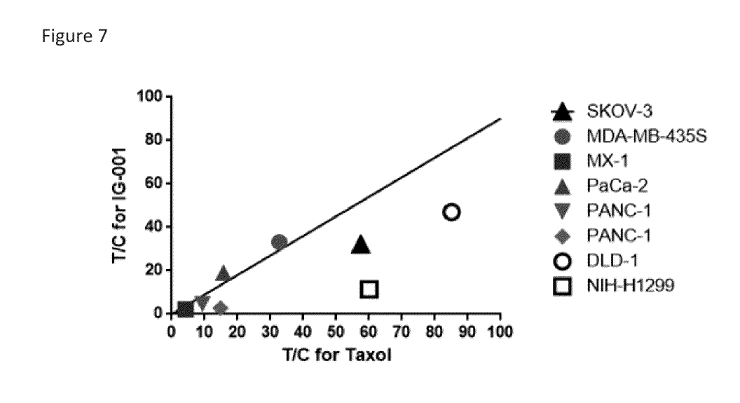

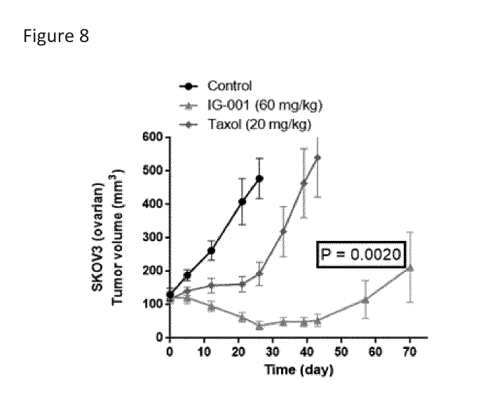

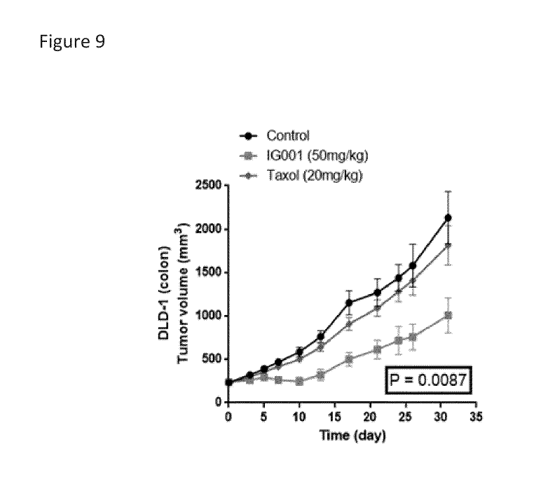

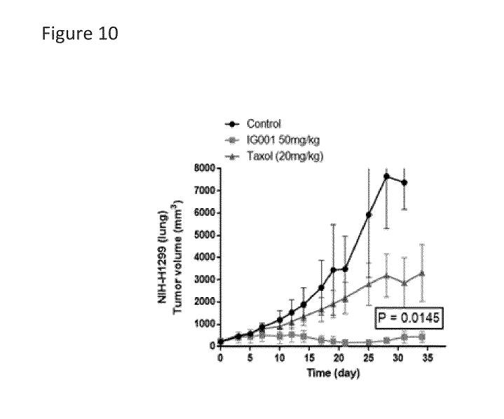

T/C using the AUC method was performed for a series of xenograft studies. The T/C of Taxol® was compared to T/C of IG-001, at equitoxic dose (20-25 mg/kg, qd×3, for Taxol® and 50-60 mg/kg, qd×3 for IG-001). Tumors resistant to Taxol® (SKOV3, DLD-1 and NIH-H1299) were more effectively treated with IG-001 (FIG. 7). Their T/C when treated with IG-001 was smaller than when treated with Taxol. ANOVA Statistic of Repeated Measurements was used to demonstrate statistical significant differences between Taxol and IG-001 for SKOV-3 (FIG. 8), DLD-1 (FIG. 9), and NIH H1299 (FIG. 10).

### Example 7

### Antitumor Activity of IG-001 Against Three Pancreatic Xenografts

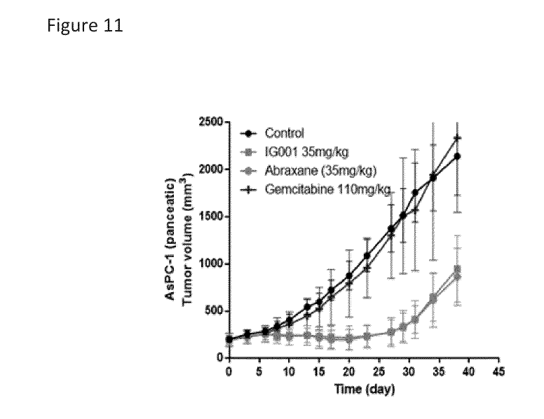

Male nude BALB/c mice (n=7/group) received a subcutaneous implantation of tumor fragments derived from the human pancreatic carcinoma cell line, AsPC-1. Tumors were allowed to reach 200 mm³ prior to initiation of intravenous (i.v.) treatments. Animals received treatments on Days 0, 3 & 6. Animals were monitored for 39 days (FIG. 11). Gemcitabine was dosed at q3d×12.

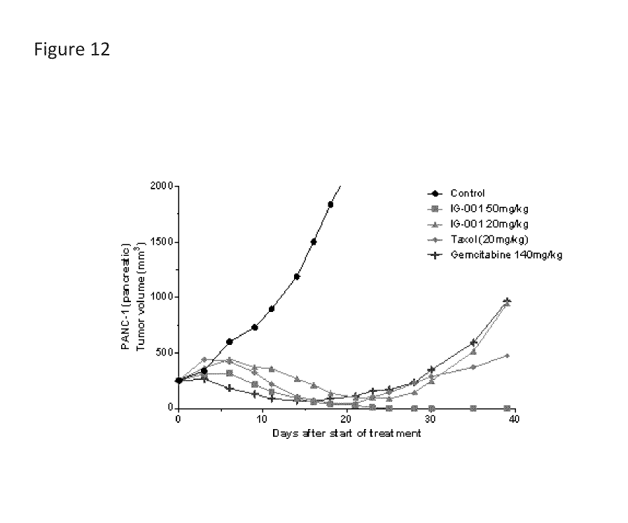

PANC-1 carcinoma: Groups of six tumor-bearing female athymic nude mice were treated with saline, Taxol® (q3d×3), Gemcitabine (q3d×12) or IG-001 (q3d×3) intravenously at the doses indicated. Tumor growth curves are shown for up to 39 days. The highest dose of IG-001 (50 mg/kg) resulted in complete remission of tumors vs. Taxol®. (FIG. 12)

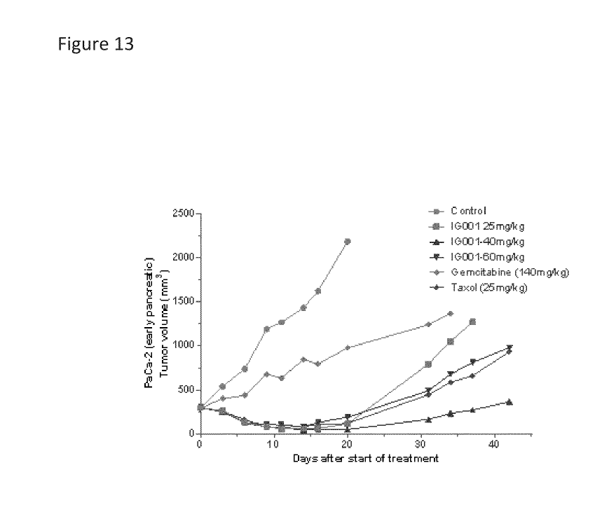

MIA PaCa-2 adenocarcinoma: Groups of seven tumor-bearing female athymic nude mice were treated with saline, Taxol® (q3d×3), Gemcitabine (q3d×12) or IG-001 (q3d×3) intravenously at the doses indicated. Tumor growth curves are shown for up to 45 days. IG-001 exhibited similar antitumor activity vs. Taxol®. (FIG. 13)

Of the three pancreatic tumor xenografts—the Taxol resistant PANC-1 was more effectively treated with IG-001 than Taxol.

Within this disclosure, any indication that a feature is optional is intended provide adequate support (e.g., under 35 U.S.C. 112 or Art. 83 and 84 of EPC) for claims that include closed or exclusive or negative language with reference to the optional feature. Exclusive language specifically excludes the particular recited feature from including any additional subject matter. For example, if it is indicated that A can be drug X, such language is intended to provide support for a claim that explicitly specifies that A consists of X alone, or that A does not include any other drugs besides X. “Negative” language explicitly excludes the optional feature itself from the scope of the claims. For example, if it is indicated that element A can include X, such language is intended to provide support for a claim that explicitly specifies that A does not include X. Non-limiting examples of exclusive or negative terms include “only,” “solely,” “consisting of,” “consisting essentially of,” “alone,” “without”, “in the absence of (e.g., other items of the same type, structure and/or function)” “excluding,” “not including”, “not”, “cannot,” or any combination and/or variation of such language.

Similarly, referents such as “a,” “an,” “said,” or “the,” are intended to support both single and/or plural occurrences unless the context indicates otherwise. For example “a dog” is intended to include support for one dog, no more than one dog, at least one dog, a plurality of dogs, etc. Non-limiting examples of qualifying terms that indicate singularity include “a single”, “one,” “alone”, “only one,” “not more than one”, etc. Non-limiting examples of qualifying terms that indicate (potential or actual) plurality include “at least one,” “one or more,” “more than one,” “two or more,” “a multiplicity,” “a plurality,” “any combination of,” “any permutation of,” “any one or more of,” etc. Claims or descriptions that include “or” between one or more members of a group are considered satisfied if one, more than one, or all of the group members are present in, employed in, or otherwise relevant to a given product or process unless indicated to the contrary or otherwise evident from the context.

Where ranges are given herein, the endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While this invention has been particularly shown and described with references to example embodiments thereof, it will be understood by those skilled in the art that the various changes in form and details may be made therein without departing from the scope of the invention encompassed by the appended claims.

Further advantages of the present immunological compositions and adjuvants of the present invention can be achieved by those skilled in the art based upon the embodiments described herein and are thus specifically within the scope of the present invention.

1-18. (canceled)

19. A method of developing a nanoparticle formulation for an effective treatment as an intravenous drug formulation for treatment of a human patient comprising:
determining one or more pharmacokinetic parameters of the drug; and
making a nanoparticle formulation which optimizes the pharmacokinetic parameters to provide an effective treatment.

20. The method of claim 19 wherein the pharmacokinetic parameters are selected from the group consisting of area under the curve (AUC), Cmax, Vd and dose proportionality.

21. The method of claim 20 wherein the AUC of the nanoparticle formulation is lower than that of solvent formulated drug.

22. The method of claim 20 wherein the Cmax of the nanoparticle is lower than that of solvent formulated drug.

23. The method of claim 20 wherein the Vd is of the nanoparticle is higher than that of solvent formulated drug.

24. The method of claim 20 wherein the dose proportionality of the nanoparticle is higher than that of solvent formulated drug.

25. The method of claim 20 wherein the AUC of the nanoparticle formulation is lower than that of solvent formulated drug, and wherein the Cmax of the nanoparticle is lower than that of solvent formulated drug, and wherein the Vd is of the nanoparticle is higher than that of solvent formulated drug.

25. The method of claim 20 wherein the drug is an intravenous therapeutic agent.

26. The method according to claim 25 wherein the drug is paclitaxel.

27. The method of claim 19 wherein the nanoparticle formulation is a polymeric micelle.

28. The method of claim 27 wherein the polymeric micelle comprises a diblock copolymer.

29. The method of claim 28 wherein the nanoparticle formulation is IG-001.

30-37. (canceled)
